(19) United States
(12) Patent Application Publication
(10) Pub. No.: US 2015/0069163 A1
(43) Pub. Date: Mar. 12, 2015

(54) MEDICAL INSTRUMENT STORAGE APPARATUS

(71) Applicant: SEIKO EPSON CORPORATION, Tokyo (JP)

(72) Inventors: Hirokazu SEKINO, Chino-shi (JP); Atsuya HIRABAYASHI, Chino-shi (JP)

(21) Appl. No.: 14/477,702

(22) Filed: Sep. 4, 2014

(30) Foreign Application Priority Data
Sep. 9, 2013 (JP) 2013-185943

(51) Int. Cl. A61M 1/00; B65H 75/44

(52) U.S. Cl. CPC A61M 1/0082; B65H 75/4439; A61M 2209/06; B65H 2701/33
USPC 242/381.1

(57) ABSTRACT

A medical instrument storage apparatus includes a storage portion configured to store a tube which constitutes part of the medical instrument therein, and a drag generating mechanism configured to generate a force acting against a force in a direction of removal of the tube stored in the storage portion from the storage portion if generated.

This application claims the benefit of Japanese Patent Application No. 2013-185943, filed on Sep. 9, 2013. The content of the aforementioned application is incorporated herein by reference in its entirety.

### BACKGROUND

1. Technical Field

The present invention relates to a medical instrument storage apparatus.

2. Related Art

In the related art, a technology disclosed in JP-A-2008-82202, for example, is known as a technology relating to a medical instrument configured to inject fluid. JP-A-2008-82202 discloses a medical instrument configured to apply pulsation to fluid by driving a piezoelectric element and perform incision and resection of an affected area by injecting the pulsed liquid to the affected area.

In the medical instrument disclosed in JP-A-2008-82202, there is a case where a biological tissue such as blood is adhered to a component which constitutes part of the medical instrument with, and hence an operation to replace the corresponding component is needed. When replacing the component with the biological tissue or the like adhered thereto in such a case, there is a demand asking for improving user convenience. In addition, in the medical instrument or apparatus relating to the medical instrument of the related art, a reduction in size, a reduction in cost, resource saving, ease of manufacture, improvement of user-friendliness are desired.

### SUMMARY

An advantage of some aspects of the invention is to solve at least part of the problems described above, and the invention can be implemented as the following forms.

(1) An embodiment of the invention provides a medical instrument storage apparatus. The medical instrument storage apparatus includes a storage portion configured to be capable of storing a tube which constitutes part of a medical instrument, and a drag generating mechanism configured to generate a force acting against a force in a direction of removal of the tube stored in the storage portion from the storage portion if generated.

In this configuration, if a user stores the tube in a state of being attached to the medical instrument in the storage portion and then moves the medical instrument in the direction of removal of the tube from the storage portion, the tube is detached from the medical instrument in the interior of the storage portion. Therefore, biological tissue or the like adhered to the tube may be restrained from flying in all directions.

(2) The medical instrument storage apparatus of the aspect described above may be configured such that the mechanism includes a rotating body, the rotating body is arranged so that an outer periphery thereof is exposed from an inner wall of the storage portion, and a rotation of the rotating body along the direction of removal of the tube from the storage portion is limited.

In this configuration, since the rotating body is allowed to rotate in a direction of storage of the tube in the storage portion, the tube may be stored in the storage portion smoothly. Furthermore, since the rotating body does not rotate in the direction of removal of the tube from the storage portion, if the user moves the medical instrument in the direction of removal of the tube from the storage portion, a frictional force is generated between the rotating body limited in rotation and the tube. Therefore, the tube may be detached easily from the medical instrument.

(3) The medical instrument storage apparatus of the aspect described above may be configured such that the number of the rotating body is plural.

In this configuration, the plurality of rotating bodies serve as guides when storing the tube in the storage portion. Furthermore, since a frictional force is generated between the plurality of rotating bodies and the tube when moving the medical instrument in the direction of removal of the tube from the storage portion, the tube may be removed further easily from the medical instrument.

(4) The medical instrument storage apparatus of the aspect described above may be configured such that the medical instrument storage apparatus further includes a projecting portion projecting from the inner wall of the storage portion at a position closer to an inlet port of the storage portion than the drag generating mechanism.

There may be a case where a supporting portion configured to support the tube is provided in the interior of the tube removable from the medical instrument. In the configuration described above, when the medical instrument is moved in the direction away from the storage portion after the tube is removed from the medical instrument, the projecting portion comes into contact with the supporting portion. Therefore, the biological tissue or the like adhered to the supporting portion may be peeled off by the projecting portion.

(5) The medical instrument storage apparatus of the aspect described above may be configured such that the projecting portion is formed of a resilient member easily deformable in a direction of storage of the tube in the storage portion and hardly deformable in the direction of removal of the tube from the storage portion.

In this configuration, the biological tissue or the like adhered to the supporting portion may be peeled off adequately.

(6) The medical instrument storage apparatus of the aspect described above may be configured such that a container is provided below the storage portion in a perpendicular direction.

In this configuration, the biological tissue or the like separated from the tube may be held in the container.

(7) The medical instrument storage apparatus of the aspect described above may be configured such that a hole is formed through the container between the storage portion and the container.

The biological tissue or the like adhered to the tube may be separated from the tube and held below the storage portion in the perpendicular direction. In the configuration described above, the biological tissue or the like held below the storage portion in the perpendicular direction may be discharged into the container through the hole.

(8) Another embodiment of the invention provides a medical instrument storage apparatus. The medical instrument storage apparatus includes a tube which constitutes part of the medical instrument, and a storage portion in which the tube is stored, wherein the storage portion is provided with a guide portion having an opening with reducing surface area along a direction away from an inlet port of the storage portion.

In this configuration, the user is allowed to perform attachment of the tube to the medical instrument again adequately after the removal of the tube from the medical instrument. In a case where the supporting portion configured to support the tube is provided in the interior of the tube which is removable from the medical instrument, the tube may be attached to the medical instrument again without making the hand contaminated with the biological tissue or the like adhered to the supporting portion.

(9) The medical instrument storage apparatus of the aspect described above may be configured such that the medical instrument storage apparatus further includes a film configured to cover the inlet port of the storage portion before use of the medical instrument storage apparatus.

In this configuration, the user is capable of determining whether or not the tube has already stored in the storage portion, or whether or not the tube having been stored in the storage portion has removed depending on whether or not the film is broken. Since entry of foreign substances or the like into the interior of the storage portion may be restrained, the foreign substances or the like may be restrained from being adhered to a tube before use.

(10) The medical instrument storage apparatus of the aspect described above may be configured such that the storage portion is removable from a main body of the medical instrument storage apparatus.

In this configuration, the main body of the medical instrument storage apparatus may be reused.

(11) Still another embodiment of the invention provides a medical instrument storage apparatus. The medical instrument storage apparatus includes a first storage portion configured to be capable of storing a tube which constitutes part of a medical instrument, a drag generating mechanism configured to generate a force acting against a force along a direction of removal of the tube stored in the first storage portion from the storage portion if generated, and a second storage portion which constitutes part of the medical instrument and stores a tube for replacement, wherein the second storage portion is provided with a guide portion having an opening with reducing surface area in a direction away from an inlet port of the storage portion.

In this configuration, an operation of removing the tube from the medical instrument and storing the tube in the first storage, and attaching a new tube stored in the second storage portion to the medical instrument may be performed quickly.

All of the plurality of components in the respective aspects of the invention described above are not necessarily essential. In order to solve part or entire problem described above or in order to achieve part or entire part of advantages described in this specification, part of the plurality of components may be modified, eliminated, and replaced by other new components as needed, and part of limited contents may be eliminated. In order to solve part or entire problem described above or in order to achieve part or entire part of advantages described in this specification, part or the entire part of technical characteristics included in one aspect of the invention described above may be combined with part or entire part of the technical characteristic included in another aspect of the invention described above to form an independent mode of the invention.

For example, one aspect of the invention can be implemented as an apparatus provided with one or more of two elements, namely, the storage portion and the drag generating mechanism. In other words, the apparatus may and may not have the storage portion. The apparatus may and may not have the drag generating mechanism. The storage portion may be configured as a storage portion configured to be capable of storing the tube which constitutes part of the medical instrument, for example. The drag generating mechanism may be configured as a drag generating mechanism configured to generate a force acting against a force in a direction of removal of the tube stored in the storage portion from the storage portion if generated.

Alternatively, another aspect of the invention can be implemented as an apparatus provided with one or more of two elements, namely, the tube and the storage portion. In other words, the apparatus may and may not have the tube. The apparatus may and may not have the storage portion. The tube may be configured as a tube which constitutes part of the medical instrument, for example. The storage portion may be configured as a storage portion configured to store the tube, for example. Alternatively, the storage portion may be provided with a guide portion having an opening with reducing surface area along a direction away from an inlet port of the storage portion.

The apparatus described above may be implemented as the medical instrument storage apparatus, for example, and may be implemented as apparatus other than the medical instrument storage apparatus. In this configuration, at least one of various problems such as a reduction in size, a reduction in cost, resource saving, ease of manufacture, and improvement of user-friendliness may be solved. Part or entire part of the technical characteristics of the respective modes of the medical instrument storage apparatus described above may be applied to the apparatus.

The invention may be implemented as various forms other than the apparatus. For example, the invention may be implemented in various forms such as a method of manufacturing a medical instrument storage apparatus, a method of storing a medical instrument, a method of detaching a medical instrument, or a method of attaching a medical instrument.

### BRIEF DESCRIPTION OF DRAWINGS

The invention will be described with reference to the accompanying drawings, wherein like numbers reference like elements.

FIG. 1 is an explanatory drawing illustrating a configuration of a medical instrument.

FIG. 2 is an explanatory drawing illustrating an internal structure of a handpiece.

FIG. 3 is an explanatory drawing illustrating an appearance of a medical instrument storage apparatus as a first embodiment.

FIG. 4 is an explanatory drawing schematically illustrating part of a cross section of the medical instrument storage apparatus as the first embodiment.

FIG. 5 is an explanatory drawing schematically illustrating part of a cross section of a medical instrument storage apparatus as a second embodiment.

FIG. 6 is an explanatory drawing schematically illustrating a cross section of a medical instrument storage apparatus as a third embodiment.

### DESCRIPTION OF EXEMPLARY EMBODIMENTS

Subsequently, embodiments of the invention will be described in the following order.

### A: Configuration of Medical Instrument

### B: First Embodiment of Medical Instrument Storage Apparatus

### C: Second Embodiment of Medical Instrument Storage Apparatus

### D: Third Embodiment of Medical Instrument Storage Apparatus

### E. Modifications

### A: Configuration of Medical Instrument

Prior to a description of a medical instrument storage apparatus as an embodiment of the invention, a medical instrument provided with a sucking tube to be stored in the medical instrument storage apparatus will be described.

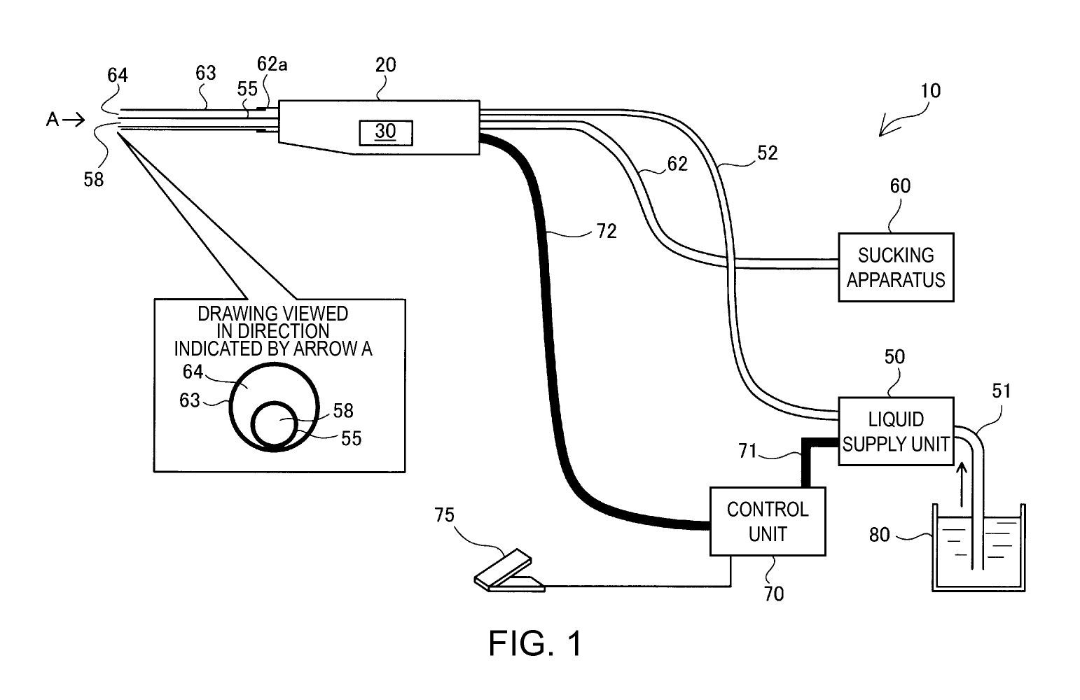

FIG. 1 is an explanatory drawing illustrating a configuration of a medical instrument 10. The medical instrument 10 of the embodiment has a function as a knife for performing incision or resection of an affected area by injecting liquid on the affected area.

The medical instrument 10 includes a handpiece 20, a liquid supply unit 50, a sucking apparatus 60, a control unit 70, and a liquid container 80. The liquid supply unit 50 and the liquid container 80 are connected by a connecting tube 51. The liquid supply unit 50 and the handpiece 20 are connected by a liquid supply flow channel 52. In the embodiment, the connecting tube 51 and the liquid supply flow channel 52 are formed of a resin.

The liquid container 80 stores normal saline solution as liquid therein. The liquid supply unit 50 supplies liquid sucked from the liquid container 80 via the connecting tube 51 to the handpiece 20 via the liquid supply flow channel 52. In the embodiment, the normal saline solution is employed as the liquid. However, the invention is not limited thereto, and various types of liquid such as aseptic water or ultrapure water may be employed.

The handpiece 20 is an instrument griped and operated by a user of the medical instrument 10. The handpiece 20 includes a pulsation generating unit 30, an injection tube 55, and an injection port 58. The liquid supplied from the liquid supply unit 50 to the handpiece 20 via the liquid supply flow channel 52 is applied with pulsation having a predetermined frequency by the pulsation generating unit 30, and is supplied to the injection tube 55. The liquid supplied to the injection tube 55 is injected as pulsed liquid from the injection port 58. The user performs incision or resection of an affected area of a patient by applying the pulsed liquid injected from the injection port 58. In the embodiment disclosed here, the injection tube 55 is formed of stainless steel. However, the injection tube 55 may be formed of other materials having at least a predetermined rigidity such as other types of metals such as brass or reinforced plastic.

The control unit 70 sends a drive signal to the pulsation generating unit 30 via a signal cable 72, and controls a flow amount of the liquid to be supplied to the pulsation generating unit 30 by controlling the liquid supply unit 50 via a control cable 71. A foot switch 75 configured to be placed near a foot of the user and operated by the user is connected to the control unit 70. When the user turns the foot switch 75 ON, the control unit 70 controls the liquid supply unit 50 to cause the pulsation generating unit 30 to supply liquid and sends the drive signal to the pulsation generating unit 30 to provide the liquid supplied to the pulsation generating unit 30 with pulsation, and then pulsed liquid is injected from the injection port 58. The expression “inject liquid in a pulsed manner” means that the liquid is injected in a state of being varied in flow amount or flow speed of the liquid to be injected. A mode of injecting the liquid in a pulsed manner includes an intermittent injection that injects the liquid by repeating injection and stop. However, what is essential is that the flow amount or the flow speed of the liquid are varied, so that the intermittent injection is not necessarily required.

The sucking apparatus 60 is an apparatus configured to suck liquid or resected tissue around the injection port 58. The sucking apparatus 60 and the handpiece 20 are connected by a sucking flow channel 62. The sucking flow channel 62 passes through the handpiece 20 and a sucking tube 63 is connected to a distal end 62a of the sucking flow channel 62. The sucking tube 63 extends to a position in the vicinity of a distal end of the injection tube 55. The injection tube 55 is inserted through the inside of the sucking tube 63. As illustrated in a drawing viewed in a direction of an arrow A in FIG. 1, a flow channel (hereinafter, referred to also as “gap flow channel”) in which liquid (resected biological tissue or the like) sucked from a suction port 64 at a distal end of the sucking tube 63 flows is defined between an outer wall of the injection tube 55 and an inner wall of the sucking tube 63. The liquid flowing into the gap flow channel from the suction port 64 is sucked into the sucking apparatus 60 via the sucking flow channel 62. In the embodiment, the sucking tube 63 is detachable from the distal end 62a of the sucking flow channel 62.

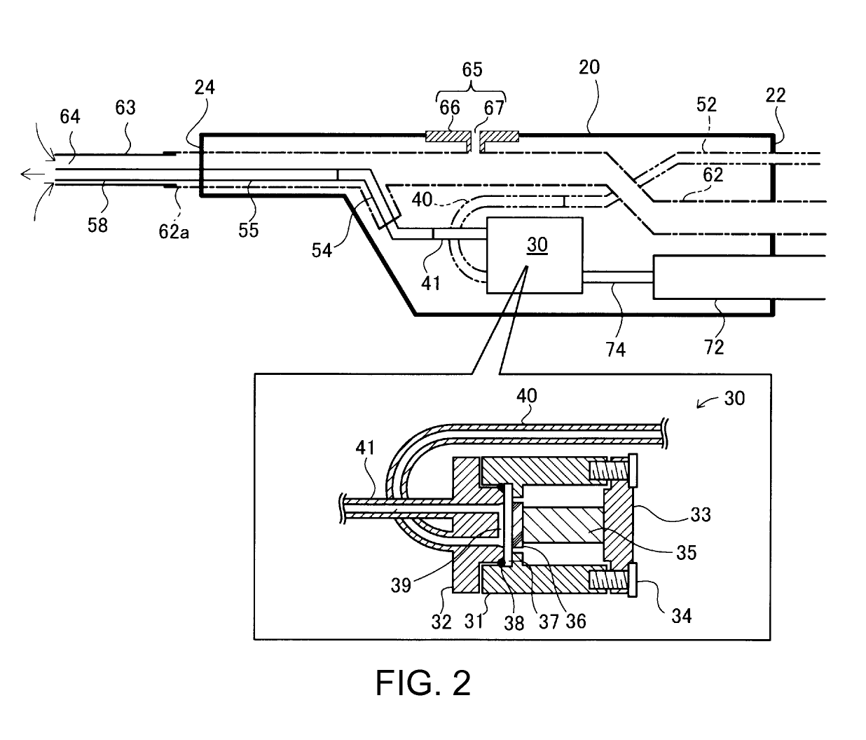

FIG. 2 is an explanatory drawing illustrating an internal structure of the handpiece 20. As described above, the pulsation generating unit 30 is stored in the interior of the handpiece 20. As illustrated in a lower portion of the drawing, the pulsation generating unit 30 includes a first case 31, a second case 32, a third case 33, a bolt 34, a piezoelectric element 35, a reinforcing plate 36, a diaphragm 37, a packing 38, an inlet flow channel 40, and an outlet flow channel 41. The first case 31 and the second case 32 are joined each other in an opposed state. The first case 31 is a cylindrical member. One of ends of the first case 31 is sealed by fixing the third case 33 with the bolt 34. The piezoelectric element 35 is disposed in a space defined in the interior of the first case 31.

The signal cable 72 is inserted from a rear end portion 22 of the handpiece 20. Two positive and negative electrode lines 74 inserted into the signal cable 72 are connected to the piezoelectric element 35 in the pulsation generating unit 30. The drive signal transmitted from the control unit 70 is sent to the piezoelectric element 35 via the electrode line 74 in the signal cable 72. The piezoelectric element 35 expands and contracts on the basis of the drive signal.

The piezoelectric element 35 is a multi-layer piezoelectric element. One of ends of the piezoelectric element 35 is secured to the diaphragm 37 via the reinforcing plate 36. The other end of the piezoelectric element 35 is secured to the third case 33. The diaphragm 37 is formed of a metallic thin film, and is secured to the first case 31 at a peripheral edge portion thereof. A liquid chamber 39 is formed between the diaphragm 37 and the second case 32. The capacity of the liquid chamber 39 is varied by driving the piezoelectric element 35.

The second case 32 is formed with the inlet flow channel 40 which liquid flows in, and the outlet flow channel 41 from which liquid flows out. The inlet flow channel 40 extends from a position deviated from a center of an end surface of the second case 32, is bent into a U-shape upward in the drawing, and extends toward the rear end portion 22 of the handpiece 20. With the inlet flow channel 40 bent as described above, a curvature of a U-shaped portion of the inlet flow channel 40 may be reduced. In addition, a total width of the inlet flow channel 40 and the pulsation generating unit 30 extending toward the rear end portion 22 may be reduced and, consequently, the handpiece 20 in which the pulsation generating unit 30 and the inlet flow channel 40 are stored may be reduced. Furthermore, with the inlet flow channel 40 extending from a distal end portion 24 side of the pulsation generating unit 30, a center of gravity of the handpiece 20 may be positioned on the distal end portion 24 side.

The liquid supply flow channel 52 is connected to the inlet flow channel 40. The injection tube 55 is connected to the outlet flow channel 41 via a connecting tube 54. The liquid supplied from the liquid supply unit 50 is supplied to the liquid chamber 39 via the liquid supply flow channel 52. When the piezoelectric element 35 vibrates at a predetermined frequency, the volume of the liquid chamber 39 is changed via the diaphragm 37, and the liquid stored therein is pressurized. The pressurized liquid passes through the outlet flow channel 41, the connecting tube 54, and the injection tube 55, and is discharged from the injection port 58.

The sucking flow channel 62 communicates from the rear end portion 22 to the distal end portion 24 of the handpiece 20. In the interior of the handpiece 20, the sucking flow channel 62 includes the connecting tube 54 and the injection tube 55 inserted therein. The sucking flow channel 62 is pulled out from the distal end portion 24 of the handpiece 20 together with the injection tube 55, and the sucking tube 63 is connected to the distal end 62a of the sucking flow channel 62. A suction force that sucks liquid or the like from the suction port 64 of the sucking tube 63 is adjustable by the sucking apparatus 60, and is adjustable by the user by operating a suction force adjusting mechanism 65 provided on the handpiece 20.

The suction force adjusting mechanism 65 includes an operating portion 66 and a hole portion 67. The hole portion 67 communicates the sucking flow channel 62 and the operating portion 66. The user opens and closes the hole portion 67 with a finger when gripping the handpiece 20. The amount of air flowing into the sucking flow channel 62 via the hole portion 67 from the outside may be adjusted by the amount of opening and closing of the hole portion 67 by the user's finger and, consequently, the suction force of the sucking flow channel 62 is adjustable. The suction force adjusting mechanism 65 is capable of achieving the function thereof desirably because the sucked material does not leak out from the hole portion 67 if being oriented upward against the gravitational force. Therefore, the suction force adjusting mechanism 65 has a function to indicate the user the up-down orientation of the handpiece 20 to be gripped.

### B: First Embodiment of Medical Instrument Storage Apparatus

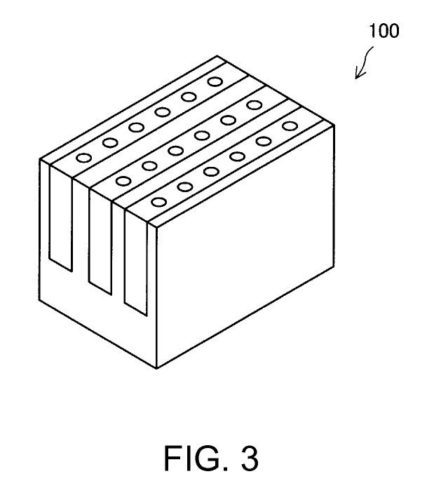

FIG. 3 is an explanatory drawing illustrating an appearance of a medical instrument storage apparatus 100 as the first embodiment of the invention. When the medical instrument 10 is used, there is a case where biological tissue or the like is adhered to the gap flow channel between an inner periphery of the sucking tube 63 and an outer periphery of the injection tube 55, and hence the suction force is lowered. Preferably, in such a case, the sucking tube 63 is removed from the medical instrument 10, and a sucking tube 63 having no biological tissue or the like adhered thereto is newly attached. The medical instrument storage apparatus 100 is configured to allow the sucking tube 63 after use having the biological tissue or the like adhered thereto to be removed from the medical instrument 10 and store the removed sucking tube 63 after use in the interior thereof.

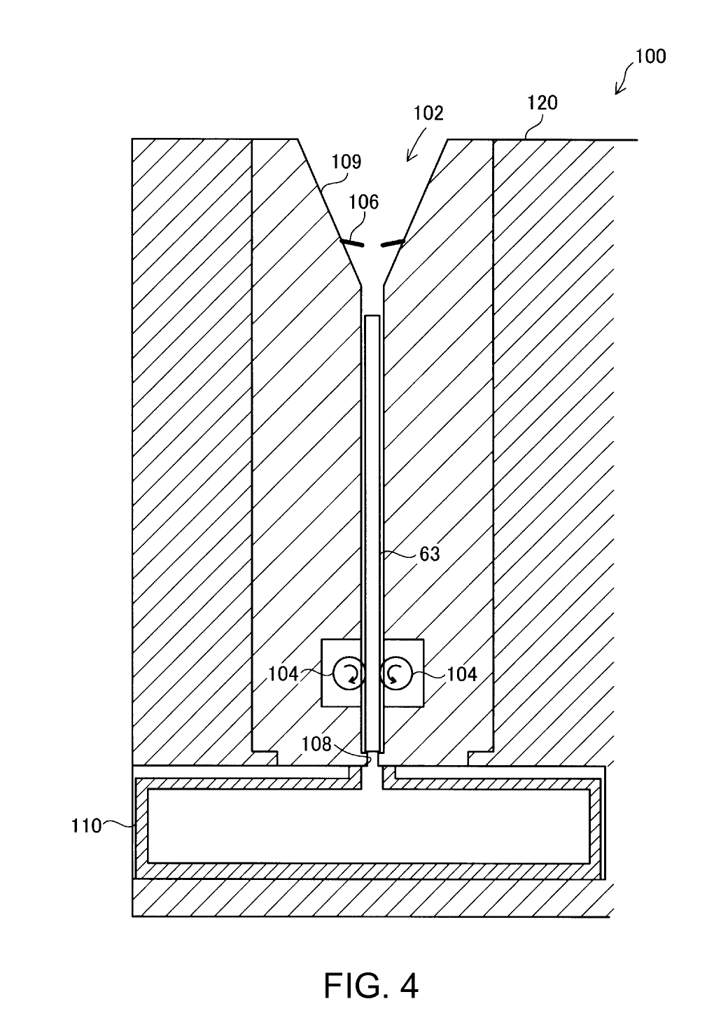

FIG. 4 is an explanatory drawing schematically illustrating part of a cross section of the medical instrument storage apparatus 100. FIG. 4 illustrates a state in which the sucking tube 63 after use is removed from the medical instrument 10 by the medical instrument storage apparatus 100 and the sucking tube 63 is stored in the interior of the medical instrument storage apparatus 100.

The medical instrument storage apparatus 100 includes a storage portion 102 and a rotating body 104. The storage portion 102 constitutes a vertically elongated space configured to be capable of storing the sucking tube 63 which constitutes part of the medical instrument 10. An outer periphery of the rotating body 104 is arranged on an inner wall of the storage portion 102. The rotation of the rotating body 104 along a direction of storage of the sucking tube 63 into the storage portion 102 is allowed. In contrast, the rotation of the rotating body 104 along a direction of removal of the sucking tube 63 from the storage portion 102 is limited. In other words, the rotating body 104 functions as a drag generating mechanism configured to generate a force acting against a force in the direction of removal the sucking tube 63 stored in the storage portion 102 from the storage portion 102 if generated.

In the first embodiment, if the user stores the sucking tube 63 in a state of being attached to the medical instrument 10 in the storage portion 102 and then moves the medical instrument 10 in the direction of removal of the sucking tube 63 from the storage portion 102, the sucking tube 63 is detached from the medical instrument 10 in the interior of the storage portion 102. Therefore, when the sucking tube 63 is removed from the medical instrument 10, biological tissue or the like adhered to the sucking tube 63 may be restrained from flying in all directions.

In particular, according to the first embodiment, since the rotating body 104 is allowed to rotate in the direction of storage of the sucking tube 63 in the storage portion 102, the sucking tube 63 may be stored in the storage portion 102 smoothly. In addition, the rotating body 104 does not rotate in the direction of removal of the sucking tube 63 from the storage portion 102. Therefore, when the user moves the medical instrument 10 in the direction of removal of the sucking tube 63 from the storage portion 102, a frictional force is generated between the rotating body 104 limited in rotation and the tube 63. Therefore, the sucking tube 63 may be removed easily from the medical instrument 10.

Furthermore, in the first embodiment, a plurality of the rotating bodies 104 are provided. Therefore, the plurality of rotating bodies 104 serve as guides when storing the sucking tube 63 in the storage portion 102. Furthermore, since the frictional force is generated between the plurality of rotating bodies 104 and the sucking tube 63 when moving the medical instrument 10 in the direction of removal of the sucking tube 63 from the storage portion 102, the sucking tube 63 may be removed further easily from the medical instrument 10. However, the rotating body 104 may be provided alone.

In the first embodiment, the storage portion 102 is provided with a projecting portion 106 on the inner wall of the storage portion 102 at a position closer to an inlet port of the storage portion 102 than the rotating bodies 104 so as to project from an entire periphery of the inner wall of the storage portion 102. As described above, the sucking tube 63 is provided with the injection tube 55 as a supporting portion configured to support the sucking tube 63 inside the sucking tube 63. Therefore, in the first embodiment, when the medical instrument 10 is moved in the direction away from the storage portion 102 after the sucking tube 63 is removed from the medical instrument 10, the projecting portion 106 comes into contact with the outer periphery of the injection tube 55. Therefore, the biological tissue or the like adhered to the outer periphery of the injection tube 55 may be peeled off by the projecting portion 106. However, the projecting portion 106 may be omitted. Also, the projecting portion 106 may be a member projecting only from part of the inner wall of the storage portion 102.

The projecting portion 106 is preferably formed of a resilient member easily deformable in the direction of storage of the sucking tube 63 in the storage portion 102 and is hardly deformable in the direction of removal of the injection tube 55 from the storage portion 102. In this configuration, biological tissue or the like adhered to the outer periphery of the injection tube 55 may be peeled off suitably when removing the injection tube 55 after the sucking tube 63 is stored in the storage portion 102. In the first embodiment, the projecting portion 106 is formed of rubber having a shape inclined in the direction of storage of the sucking tube 63 in the storage portion 102.

Furthermore, in the first embodiment, a hole 108 is provided perpendicularly below the storage portion 102, and a container 110 is provided perpendicularly below the hole 108. The biological tissue or the like adhered to the sucking tube 63 may be separated from the sucking tube 63 and stay perpendicularly below the storage portion 102. In the first embodiment, since the biological tissue or the like staying perpendicularly below the storage portion 102 may be discharged into the container 110 via the hole 108, the biological tissue or the like separated from the sucking tube 63 may be stored in the container 110. However, the hole 108 and the container 110 may be omitted. Polymer absorber which is capable of absorbing liquid such as biological tissue or the like may be provided in the interior of the container 110, or instead of the container 110.

Furthermore, in the first embodiment, the storage portion 102 is formed of a separate member from a body 120 of the medical instrument storage apparatus 100, and is removable from the body 120 of the medical instrument storage apparatus 100. Therefore, according to the first embodiment, the body 120 of the medical instrument storage apparatus 100 may be reused. However, the storage portion 102 and the body 120 of the medical instrument storage apparatus 100 may be formed integrally.

Furthermore, in the first embodiment, the opening of the inlet port of the storage portion 102 is largely extended, and a guide portion 109 having an opening with reducing surface area in a direction away from the inlet port of the storage portion 102. Therefore, the user is capable of store the sucking tube 63 easily in the storage portion 102 along the guide portion 109.

In this manner, in the first embodiment, since the drag generating mechanism configured to generate a force acting against a force in the direction of removal the sucking tube 63 stored in the storage portion 102 from the storage portion 102, if generated, is provided, the sucking tube 63 is removed from the medical instrument 10 in the interior of the storage portion 102. Therefore, biological tissue or the like adhered to the sucking tube 63 may be restrained from flying in all directions.

### C: Second Embodiment of Medical Instrument Storage Apparatus

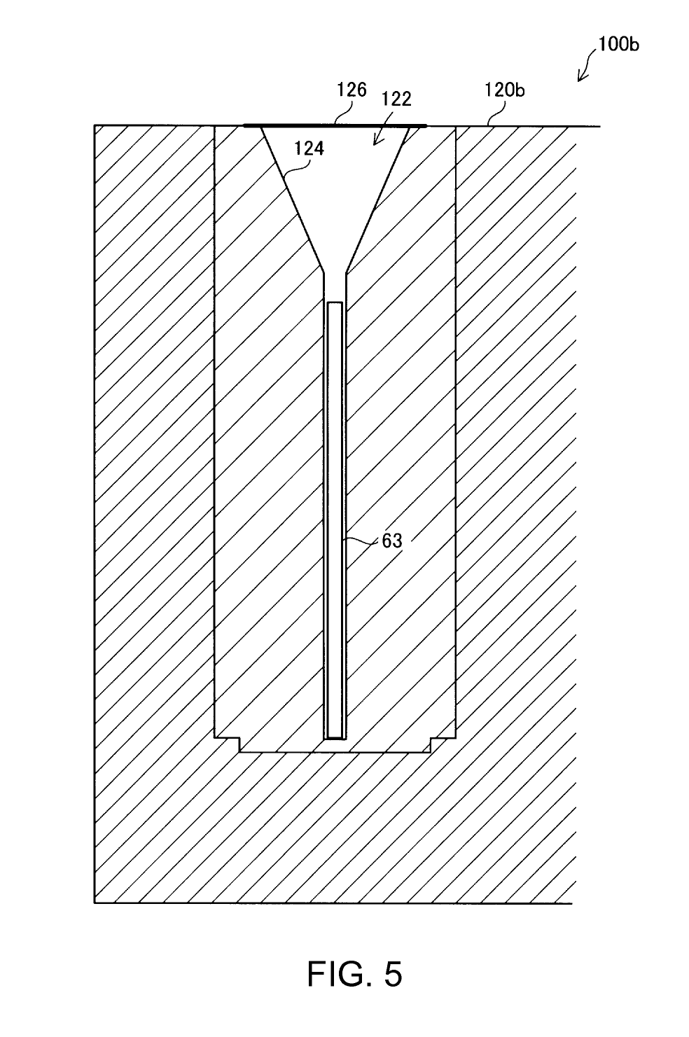

FIG. 5 is an explanatory drawing schematically illustrating part of a cross section of a medical instrument storage apparatus 100b as a second embodiment. The medical instrument storage apparatus 100b is an apparatus configured to attach a sucking tube 63 on which no biological tissue or the like is adhered newly on the medical instrument 10 in a state in which the sucking tube 63 after use having the biological tissue or the like adhered thereto is removed.

The medical instrument storage apparatus 100b includes a storage portion 122 which defines a vertically elongated space. In the storage portion 122, a sucking tube 63 before use and having no biological tissue or the like adhered thereto is stored.

In the second embodiment, the storage portion 122 is provided with a guide portion 124 having an opening with reducing surface area in a direction away from an inlet port of the storage portion 122. The user inserts the distal end of the injection tube 55 of the medical instrument 10 in a state in which the sucking tube 63 is removed into the interior of the storage portion 122 along the guide portion 124. Accordingly, the sucking tube 63 before use and stored in the storage portion 122 is newly attached to the medical instrument 10.

Therefore, according to the second embodiment, the user is allowed to perform an operation of attaching a sucking tube 63 to the medical instrument 10 newly without contaminating the hand with the biological tissue or the like adhered to the injection tube 55.

In addition, in the second embodiment, the medical instrument storage apparatus 100b is provided with a film 126 configured to cover the inlet port of the storage portion 122. In the second embodiment, the film 126 is formed of polyethylene.

When attaching the sucking tube 63 before use to the medical instrument 10, the user presses the distal end of the injection tube 55 against the film 126 to break the film 126. After the film 126 is broken, the user inserts the injection tube 55 of the medical instrument 10 as-is into the storage portion 122, and attaches the sucking tube 63 before use and stored in the interior of the storage portion 122 to the medical instrument 10. The user may break the film 126 by using a blade such as a cutter instead of the distal end of the injection tube 55.

Therefore, according to the second embodiment, the user may determine whether or not the sucking tube 63 stored in the storage portion 102 has been removed depending on whether or not the film 126 is broken. Since entry of foreign substance or the like into the interior of the storage portion 102 may be restrained, adhesion of the foreign substance or the like to the sucking tube 63 before use may be restrained.

In the second embodiment, the storage portion 122 is formed of a member separate from a body 120b of the medical instrument storage apparatus 100b, and may be removed from the main body of the medical instrument storage apparatus 100. Therefore, in the second embodiment, the body 120b of the medical instrument storage apparatus 100b may be reused. However, the storage portion 122 and the body 120b of the medical instrument storage apparatus 100b may be formed integrally.

### D: Third Embodiment of Medical Instrument Storage Apparatus

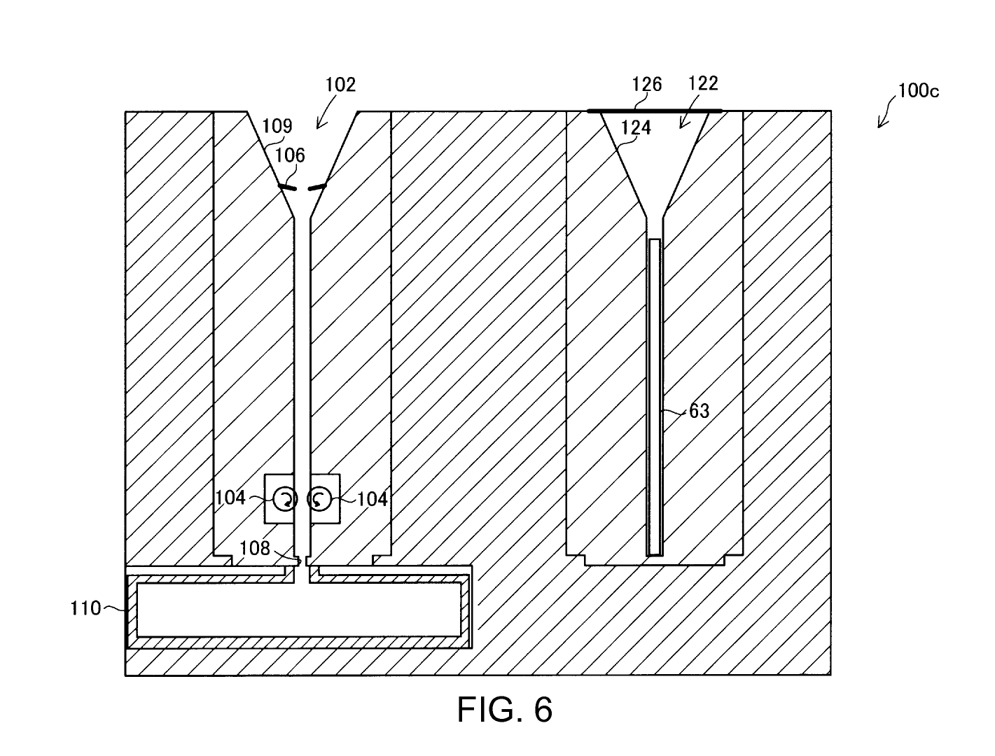

FIG. 6 is an explanatory drawing illustrating a cross section of a medical instrument storage apparatus 100c as a third embodiment. The medical instrument storage apparatus 100c of the third embodiment has a structure in which the medical instrument storage apparatus 100 of the first embodiment and the medical instrument storage apparatus 100b of the second embodiment are stored in the same housing. For example, the medical instrument storage apparatus 100c includes the storage portion 102 (first embodiment) configured to store the sucking tube 63 after use and having the biological tissue or the like adhered thereto, and the storage portion 122 (second embodiment) including the sucking tube 63 before use and having no biological tissue or the like adhered thereto. Other configurations of the medical instrument storage apparatus 100c are also the same as those of the first embodiment and the second embodiment.

Therefore, according to the third embodiment, an operation of removing the sucking tube 63 after use from the medical instrument 10 and attaching the sucking tube 63 before use to the medical instrument 10 may be performed quickly.

### E. Modifications

The invention is not limited to the embodiments or examples described above, and various modes may be implemented without departing the scope of the invention. For example, the following modifications may be applied.

### Modification 1

In the first embodiment, a film configured to cover the inlet port of the storage portion 102 may be provided in the same manner as the second embodiment. In this configuration, the user may determine whether or not the sucking tube 63 after use has been stored in the storage portion 102 depending on whether or not the film is broken. In the storage portion 102 of the third embodiment, the film configured to cover the inlet port of the storage portion 102 may be formed in the same manner.

### Modification 2

In the first and the third embodiments, another drag generating mechanism may be provided instead of the rotating bodies 104. For example, a narrowed portion reduced in diameter gradually from an outer diameter of the sucking tube 63 may be provided at a portion apart from the inlet port of the storage portion 102 as the drag generating mechanism.

### Modification 3

In the first embodiment, only one storage portion 102 may be provided on the medical instrument storage apparatus 100. In the second embodiment, in the same manner, only one storage portion 122 may be provided on the medical instrument storage apparatus 100b.

### Modification 4

In the respective embodiments described above, attachment and detachment of the sucking tube 63 are performed by the medical instrument storage apparatus. However, attachment and detachment of the injection tube 55 may be performed by the medical instrument storage apparatus instead of the sucking tube 63 in the medical instrument 10 which is not provided with the sucking tube 63.

The invention is not limited to the embodiments, the examples, and the modifications described above, and various modes may be implemented without departing the scope of the invention. For example, technical characteristics in the embodiments, the examples, and the modifications corresponding to the technical characteristics in the respective embodiments in the respective modes described in the paragraph of the summary may be replaced or combined as needed in order to solve part or entire problem described above or in order to achieve part or entire part of the above-described advantages. The technical characteristics may be eliminated as needed unless otherwise specified to be essential in the specification.

What is claimed is:

1. A medical instrument storage apparatus, comprising:
a storage portion configured to store a tube which constitutes part of a medical instrument, and
a drag generating mechanism configured to generate a force acting against a force in a direction of removal of the tube stored in the storage portion from the storage portion if generated.

2. The medical instrument storage apparatus according to claim 1,
wherein the drag generating mechanism includes a rotating body,
the rotating body is arranged so that an outer periphery thereof is exposed from an inner wall of the storage portion, and
a rotation of the rotating body along the direction of removal of the tube from the storage portion is limited.

3. The medical instrument storage apparatus according to claim 2,
wherein a plurality of the rotating bodies are provided.

4. The medical instrument storage apparatus according to claim 1, further comprising:
a projecting portion projecting from an inner wall of the storage portion at a position closer to an inlet port of the storage portion than the drag generating mechanism.

5. The medical instrument storage apparatus according to claim 4,
wherein the projecting portion is formed of a resilient member easily deformable in a direction of storage of the tube in the storage portion and hardly deformable in the direction of removal of the tube from the storage portion.

6. The medical instrument storage apparatus according to claim 1, further comprising:
a container provided perpendicularly below the storage portion.

7. The medical instrument storage apparatus according to claim 6, comprising:
a hole communicating with the container provided between the storage portion and the container.

8. A medical instrument storage apparatus, comprising:
a tube constituting part of a medical instrument; and
a storage portion in which the tube is stored,
wherein the storage portion is provided with a guide portion having an opening with reducing surface area along a direction away from an inlet port of the storage portion.

9. The medical instrument storage apparatus according to claim 1, further comprising:
a film configured to cover the inlet port of the storage portion before use of the medical instrument storage apparatus.

10. The medical instrument storage apparatus according to claim 8, further comprising:
a film configured to cover the inlet port of the storage portion before use of the medical instrument storage apparatus.

11. The medical instrument storage apparatus according to claim 1,
wherein the storage portion is removable from the main body of the medical instrument storage apparatus.

12. The medical instrument storage apparatus according to claim 8,
wherein the storage portion is removable from the main body of the medical instrument storage apparatus.

13. A medical instrument storage apparatus, comprising:
a first storage portion configured to be capable of storing a tube which constitutes part of a medical instrument,
a drag generating mechanism configured to generate a force acting against a force in a direction of removal of the tube stored in the first storage portion from the storage portion if generated, and
a second storage portion configured to store a tube which constitutes part of the medical instrument and stores a tube for replacement,
wherein the second storage portion is provided with a guide portion having an opening with reducing surface area along a direction away from an inlet port of the storage portion.
